(19) United States
(12) Patent Application Publication
(10) Pub. No.: US 2025/0283292 A1
(43) Pub. Date: Sep. 11, 2025

(54) MONITORING DEVICES AND ITS INSTALLATION METHODS FOR SEABED SOIL LIQUEFACTION

(71) Applicant: Chao Dao Environment and Energy Ltd., Kaohsiung City (TW)

(72) Inventors: Yu-Hsiu TSENG, Kaohsiung City (TW); Kai Jun CHONG, Kaohsiung City (TW)

(21) Appl. No.: 19/041,331

(22) Filed: Jan. 30, 2025

(30) Foreign Application Priority Data
Mar. 11, 2024 (TW) 113108794

(51) Int. Cl. E02D 33/00; G01N 33/24

(52) U.S. Cl. CPC E02D 33/00; G01N 33/246; E02D 2600/10; E02D 2600/20

(57) ABSTRACT

A seabed soil liquefaction monitoring device for installation on a seabed soil to monitor liquefaction status, includes a first monitoring unit exposed above the seabed soil, including a data logger for recording the measurement data of a soil pressure transducer and a piezometer; an accelerometer for measuring the acceleration of the device under the action an earthquake; and a second monitoring unit fixed to the first monitoring unit and extending into the seabed soil, and including a lower cover with a first opening and at least one second opening; a suction bucket to penetrate the seabed soil, including the soil pressure transducer and the piezometer; wherein the first opening passes through an electrical conduit to accommodate signal lines and power lines of the seabed soil liquefaction monitoring device, while the at least one second opening passed through a drainage pipe to drain water from the suction bucket.

### CROSS-REFERENCE TO RELATED APPLICATION

This application claims the priority benefit of Taiwan application No. 113108794, filed 2024 Mar. 11. The entirety of each of the above-mentioned patent applications is hereby incorporated by reference herein and made a part of this specification.

### TECHNICAL FIELD

This invention relates to a seabed soil liquefaction monitoring device and its installation method, mainly through a workboat to place the monitoring device in the seabed area to monitor the liquefaction status of the seabed soil, as well as the method of transmitting monitoring data.

### BACKGROUND

Currently, there have been no actual cases of monitoring soil liquefaction in marine environment, only examples such as soil liquefaction monitoring devices and installation methods for terrestrial areas. Referring to the soil liquefaction monitoring device and method proposed in Taiwanese Patent 1821140, as shown in FIG. 6, the terrestrial soil liquefaction monitoring device and method use a mini suction bucket in combination with a piezometer and soil pressure transducer. Through a suction installation method, the equipment is prevented from sinking into or floating up from liquefied soil when soil liquefaction occurs. However, the terrestrial soil liquefaction monitoring device cannot be directly applied to the marine environment, the main reasons for which are: Due to the deep water environment of offshore marine areas being too far from the shore, the monitoring device cannot directly obtain power supply and telecommunication signals like terrestrial soil liquefaction monitoring devices, therefore it needs to be equipped with independent power supply systems and real-time signal transmission systems in order to meet the objective of transmitting measurement data in real-time for remote monitoring; The main cause of terrestrial soils liquefaction is primarily attributed to seismic loads. Since the dense seismic network on terrestrial areas, the seismic and monitoring data from these seismic stations could be cross-referenced and confirmed. However, the characteristics of the marine environment are complex, seabed soil can experience excess pore water pressure triggered by various causes beyond seismic loads alone. The repeated impacts of extreme wave loads on the seabed can also induce this situation. Due to the lack of seismic stations in offshore areas, it is challenging to directly obtain information on whether an earthquake has occurred on the seabed and its magnitude. This makes it difficult to compare and verify the measurement data, and to determine whether the excess pore water pressure originates from the action of extreme wave loads or seismic loads. Consequently, the seabed soil liquefaction monitoring device needs to be specially designed to consider how to distinguish the cause behind the excitation of excess pore water pressure.

Therefore, this application proposes a structure design and installation method for a soil liquefaction monitoring device in marine environments, solving the problem of the monitoring device in marine environments being unable to directly obtain power supply and telecommunication signals, and further addressing the issue of being unable to compare and analyze measurement data with seismic data in such marine environments.

### SUMMARY

A seabed soil liquefaction monitoring device designed for installation on a seabed soil to monitor its liquefaction status, comprising: a first monitoring unit exposed above the seabed soil, comprising: a data logger for recording the measurement data of a soil pressure transducer and a piezometer; an accelerometer for measuring the acceleration of the seabed soil liquefaction monitoring device under the action of an earthquake; and a second monitoring unit fixed to the first monitoring unit and extending into the seabed soil, the second monitoring unit comprising: an lower cover with a first opening and at least one second opening; a suction bucket to penetrate the seabed soil, comprising: the soil pressure transducer for measuring soil stress of the seabed soil; and the piezometer for measuring pore water pressure of the seabed soil; wherein the first opening is used for passing through an electrical conduit to accommodate signal lines and power lines of the seabed soil liquefaction monitoring device, while the at least one second opening is used for passing through a drainage pipe to drain water from the suction bucket.

The seabed soil liquefaction monitoring device according to this invention, wherein the suction bucket further comprises multiple probes that extend into the seabed soil for connecting the accelerometer and transmitting seismic wave data.

The seabed soil liquefaction monitoring device according to this invention, the first monitoring unit is a sealed chamber used to prevent external seawater from entering.

The seabed soil liquefaction monitoring device according to this invention, the at least second opening are set to three openings and evenly spaced around the lower top cover, along with the first opening.

The seabed soil liquefaction monitoring device according to this invention, wherein, the piezometer and soil pressure transducer are installed inside the suction bucket and positioned near the first opening.

The seabed soil liquefaction monitoring device according to this invention, wherein, the first monitoring unit further comprising a battery, which provides electric power to the data logger, accelerometer, soil pressure transducer, and piezometer. The battery can store enough electricity for at least five days' use while sunless.

The seabed soil liquefaction monitoring device according to this invention, further comprising a buoy system connecting the electrical conduit and drainage pipe, the buoy system comprising: a signal transmission system, used for transmitting the data measured from the seabed soil liquefaction device for remote monitoring; and a solar photovoltaic system installed on the buoy system, used for generating and transmitting electricity to the battery through the electrical conduit.

The seabed soil liquefaction monitoring device according to this invention, wherein, the first monitoring unit is a first cylindrical shape, and the bottom surface of the first monitoring unit is smaller than the top surface of the second monitoring unit in order to accommodate the first opening and at least one second opening.

The seabed soil liquefaction monitoring device according to this invention, wherein, the first opening and at least one second opening are positioned opposite each other on both sides of the first monitoring unit.

The seabed soil liquefaction monitoring device according to this invention, wherein, the second monitoring unit is a second cylindrical shape, and the circular base area of the second cylindrical shape is larger than the circular base area of the first cylindrical shape of the first monitoring unit, in order to maintain the structural stability of the seabed soil liquefaction monitoring device.

The seabed soil liquefaction monitoring device according to this invention, wherein, the height of one side of the cylindrical shape of the second monitoring unit is greater than the height of one side of the cylindrical shape of the first monitoring unit, in order to maintain a structural stability of the seabed soil liquefaction monitoring device.

The present invention discloses a method for installing a seabed soil liquefaction monitoring device, which is used to penetrate the seabed soil liquefaction monitoring device into the seabed soil, the seabed soil liquefaction monitoring device comprising a first monitoring unit and a second monitoring unit, the second monitoring unit comprising a suction bucket formed by a lower top cover and a lower side wall, the installation method comprising: the first monitoring unit is secured to a crane on a workboat by using a rope; securing one end of an electrical conduit to the workboat, while the other end of the electrical conduit passing through the first opening of the lower top cover and extending into the suction bucket; connecting one end of a drainage pipe to a pumping device on the workboat, while the other end of the drainage pipe passing through at least one second opening on the lower top cover and extending into the suction bucket; opening the valve of the drainage pipe; controlling the crane to lower the seabed soil liquefaction monitoring device down to the surface of the seabed; releasing the tension of the rope to partially penetrate seabed soil liquefaction monitoring device into the seabed soil; and controlling the pumping device, the water within the suction bucket could be drained through the drainage pipe, allowing the suction bucket to penetrate almost completely into the seabed soil.

The present invention discloses another method for installing a seabed soil liquefaction monitoring device, which is used to penetrate the device into the seabed soil, the seabed soil liquefaction monitoring device comprising a first monitoring unit and a second monitoring unit; the second monitoring unit comprising a lower top cover and a suction bucket, the installation method includes: connecting a drill rod of a workboat to the first monitoring unit using a rod; securing one end of an electrical conduit to the workboat, while the other end of the electrical conduit passing through a first opening on the lower top cover and extending into the suction bucket; connecting one end of a drainage pipe to a pumping device on the workboat, while the other end of the drainage pipe passing through at least one second opening in the lower top cover and extending into the suction bucket; opening the valve of the drainage pipe; controlling the drill rod to lower the seabed soil liquefaction monitoring device down to the surface of the seabed soil; and controlling the drill rod to provide a necessary resistance to penetrate the suction bucket into the seabed soil, allowing the suction bucket to penetrate almost completely into the seabed soil.

The method for installing the seabed soil liquefaction monitoring device according to this invention, further comprising: when the seabed soil liquefaction monitoring device is almost completely penetrated into the seabed soil, closing the valve of the drainage pipe to maintain the suction pressure inside the suction bucket, then connecting the drainage pipe and the electrical conduit to a buoy system, by positioning the buoy system, the installation location of the monitoring device can still easily be located and identified in the future.

The method for installing the seabed soil liquefaction monitoring device according to this invention, further comprising: when the seabed soil liquefaction monitoring device is almost completely penetrated into the seabed soil, injecting a highly fluid and fast-setting material into the drainage pipe, then connecting the drainage pipe and the electrical conduit to a buoy system, by positioning the buoy system, the installation location of the monitoring device can still easily be identified and located in the future.

The method for installing the seabed soil liquefaction monitoring device according to this invention, the buoy system comprising a signal transmission system, which is used for transmitting the data measured by the seabed soil liquefaction monitoring device timely for remote monitoring; and a solar power system, which is installed on the buoy system for generating and transmitting electricity to the battery through the electrical conduit.

The method for installing the seabed soil liquefaction monitoring device according to this invention, further comprising placing multiple erosion protection works around the seabed soil liquefaction monitoring device to reduce the possibility of local erosion caused by sea currents.

### BRIEF DESCRIPTION OF THE DRAWINGS

FIG. 1 illustrates the seabed soil liquefaction monitoring device of the present invention.

FIG. 2 illustrates the inner structure of the first monitoring device of the present invention.

FIG. 3. illustrates the inner structure of the second monitoring device of the present invention.

FIG. 4A to 4E illustrates a method for installing the seabed soil liquefaction monitoring device according to this invention.

FIG. 5 illustrates another method for installing the seabed soil liquefaction monitoring device according to this invention.

FIG. 6 illustrates a prior art schematic diagram for a terrestrial soil liquefaction monitoring device and method.

### DETAILED DESCRIPTION

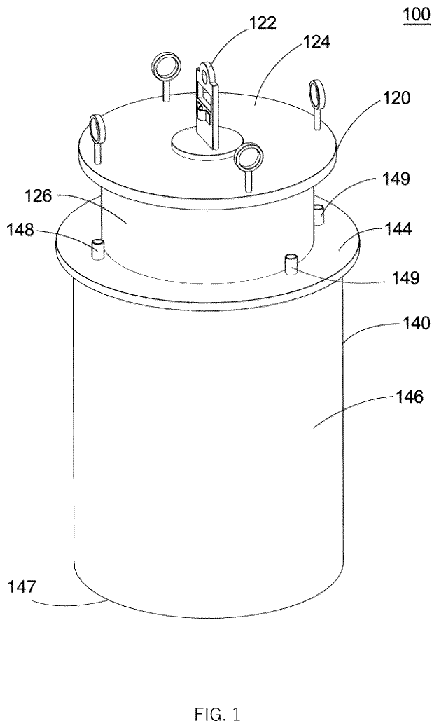

Due to the unique environment of the marine environment, in addition to seismic loads, the possible causes of excess pore water pressure in the seabed soil can be triggered by the repetitive action of extreme wave loads. Therefore, in order to identify the causes of excess pore water pressure in the seabed soil detected by the monitoring device, please refer to FIG. 1 for the seabed soil liquefaction monitoring device 100 of the present invention, which is used to detect the liquefaction status of the seabed soil 900 where it is installed. The monitoring device 100 includes a first monitoring unit 120 and a second monitoring unit 140. The first monitoring unit 120 is a sealed cavity formed by the upper cover 124, the upper side wall 126, and the lower cover 144, which can prevent seawater from entering. It includes a simple joint 122 set on the upper cover 124 to connect the ropes or rods used for subsequent placement of this seabed soil liquefaction monitoring device 100. The second monitoring unit 140 is a suction bucket 147 formed by the lower cover 144 and the lower side wall 146 with a downward opening. The lower cover 144 has a first opening 148 for the passage of electrical conduit, and at least one second opening 149, for example, three drainage pipes can be connected individually. In the preferred configuration, these three second openings 149 are uniformly arranged around the lower cover 144, so that the suction bucket can have balanced downward pressure distribution during the process of removing water and penetration into the seabed soil. When the suction bucket 147 is slightly off balance during the penetration process, the pumping rate of the second opening 149 on the higher side can be increased to accelerate the penetration speed of that side, dynamically adjusting the suction bucket 147 to return to a balanced state. The area surrounded by the upper side wall 126 of the first monitoring unit 120 is smaller than the area surrounded by the lower side wall 146 of the second monitoring unit 140, and the height of the upper side wall 126 of the first monitoring unit 120 is also smaller than the height of the lower side wall 146 of the second monitoring unit 140. This design can effectively enhance the structural stability of the monitoring device 100 and ensure that the penetration of the monitoring device 100 in subsequent installation steps is less affected by sea current flow and has less angular and positional deviation.

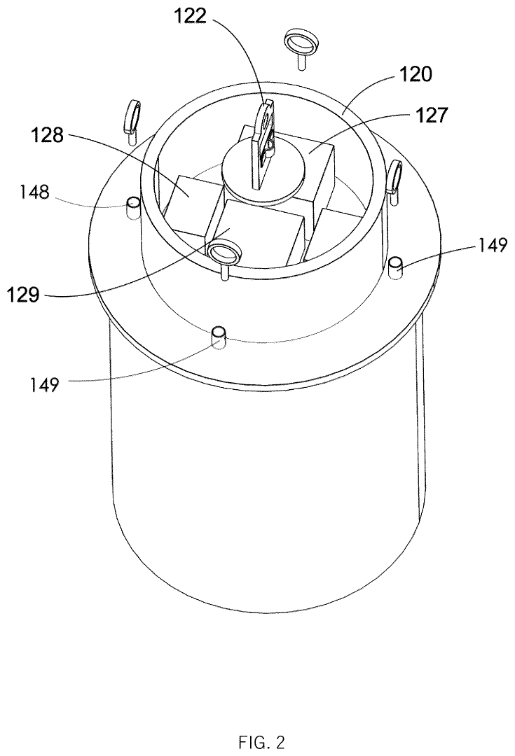

Please refer to FIG. 2 for the internal structural diagram of the first monitoring unit 120. It includes an accelerometer 129, used to measure the acceleration of the seabed soil liquefaction monitoring device 100 under seismic action. A data logger 127 is used to record the measurement signals from the soil pressure transducer 142 and the piezometer 143. A battery 128 is used to provide power to the accelerometer 129, data logger 127, soil pressure transducer 142, and piezometer 143, especially in unstable weather conditions at sea. If the subsequent storage of electricity generated by the solar power system is used, it is ideal to have a storage capacity for at least five days' use while sunless. The first monitoring unit 120 is mainly used to detect nearby seismic waves and can determine whether the excitation of pore water pressure in the soil is caused by an earthquake based on the acceleration measured by the accelerometer 129. The circuits or power lines of the accelerometer 129, data logger 127, and battery 128 can enter the second monitoring unit 140 (not shown in the figure) first and then be transmitted or received from the outside through its first opening 148 and the electrical conduit 300 passing through it. However, the airtightness of the first monitoring unit 120 must be maintained in this structure.

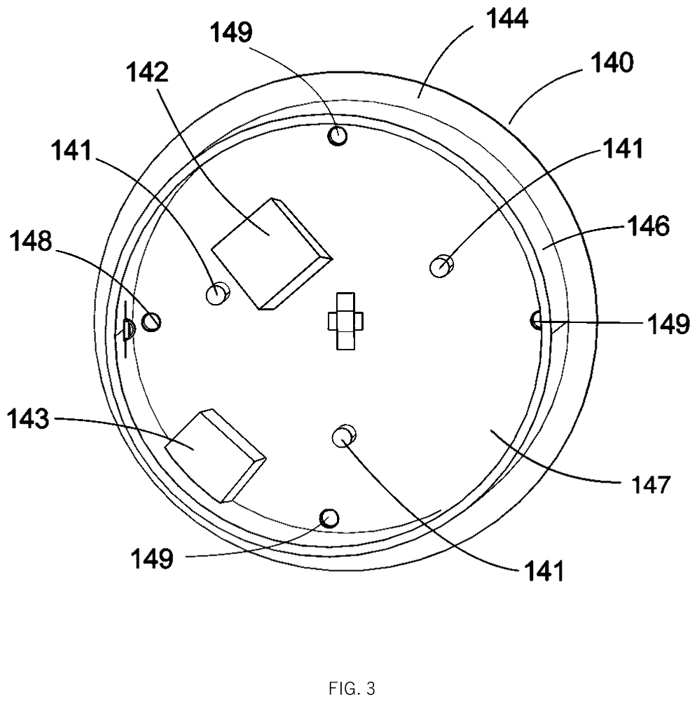

Please refer to FIG. 3 for the internal structural diagram of the second monitoring unit 140. It includes a soil pressure transducer 142, which is installed on the lower surface of the lower cover 144 to measure the soil stress of the seabed soil. A piezometer 143 is installed on the inner surface of the lower side wall 146 to measure the pore water pressure of the seabed soil. The information collected by the above-mentioned equipment can be used to determine whether liquefaction has occurred in the seabed soil. The soil pressure transducer 142 and the piezometer 143 can be placed close to the first opening 148 and at a distance from the second opening 149. This is mainly because the first opening 148 is primarily used for the passage of the electrical conduit 300, making it easier to arrange the signal line layout of the soil pressure transducer 142 and the piezometer 143. When setting up the suction bucket 147 of the second monitoring unit 140, there may be a gap between the lower cover 144 and the seabed. Therefore, multiple probes 141 are installed inside the second monitoring unit 140, which are structurally directly connected to the accelerometer 129 of the first monitoring unit 120. The ends of the multiple probes 141 can be inserted into the seabed soil, allowing the accelerometer 129 to accurately measure seismic data without being affected by the gap.

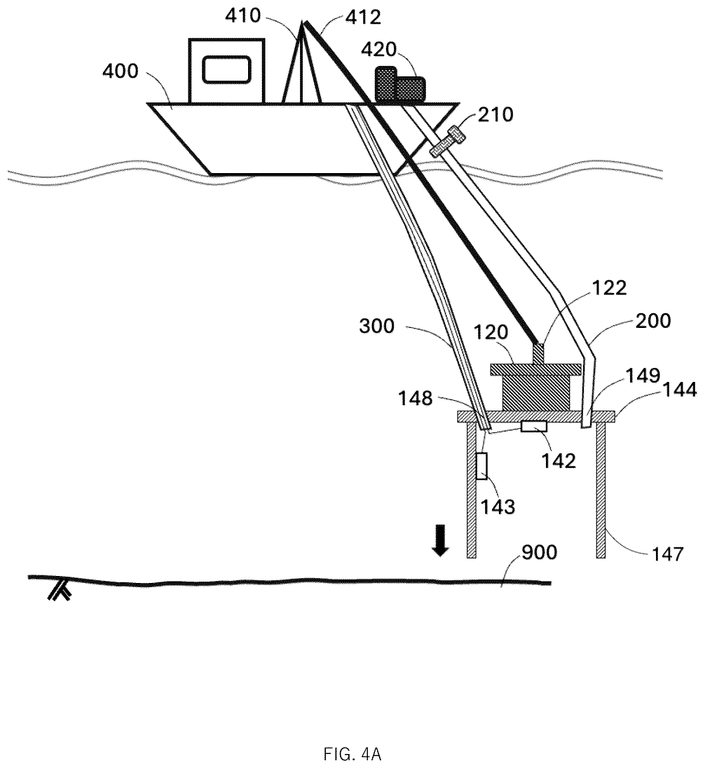

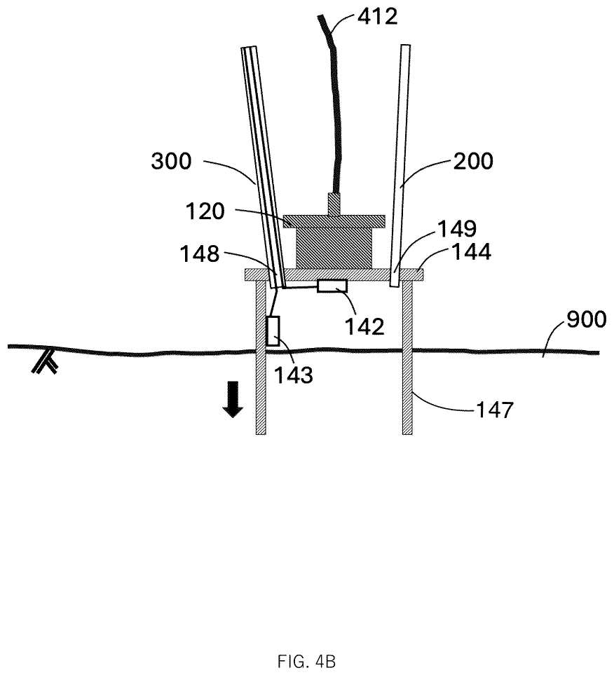

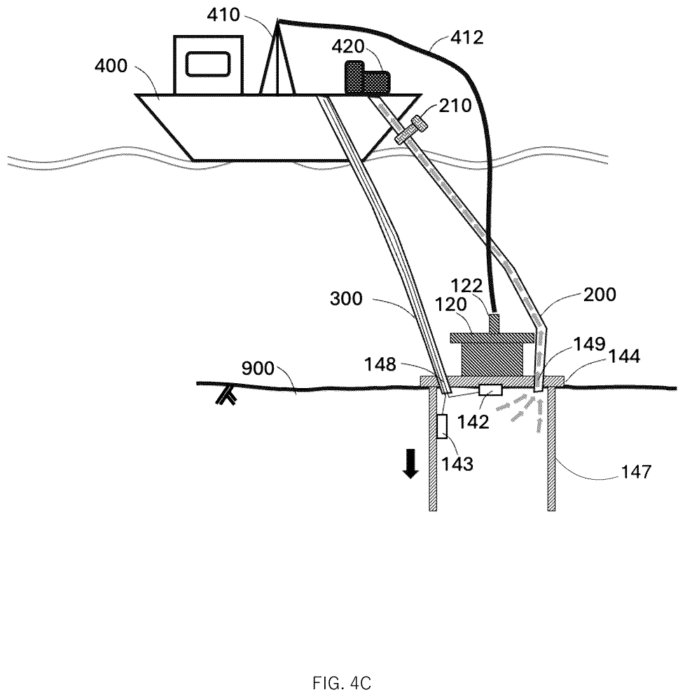

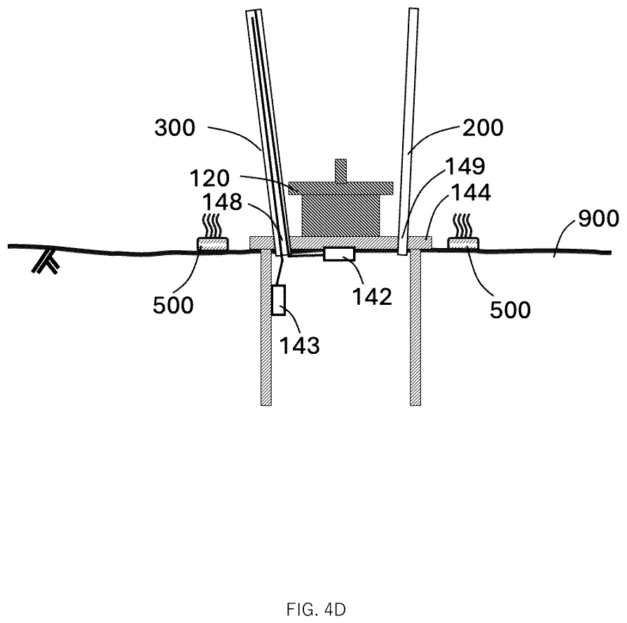

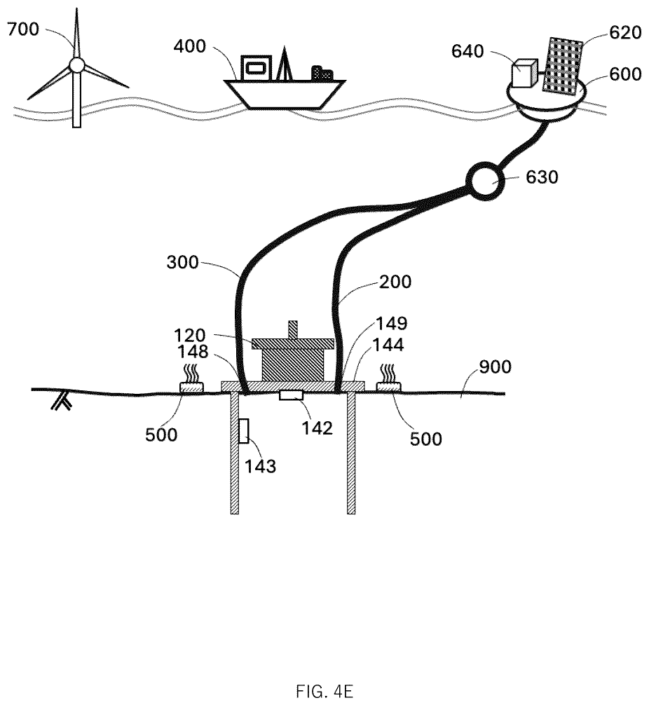

Next, please refer to FIGS. 4A to 4E for the installation method of the seabed soil monitoring device 100 of the present invention. First, please refer to FIG. 4A. Conduct a side-scan sonar survey (not shown in the figure) at the selected installation location on the seabed soil 900. Secure the monitoring device 100 to the crane 410 on the workboat 400 using a rope 412. Fix one end of the electrical conduit 300 on the workboat 400, and pass the other end through the first opening 148 of the lower cover 144 and into the suction bucket 147 of the second monitoring unit 140. Connect one end of the drainage pipe 200 to a pumping device 420 on the workboat 400, and pass the other end of the drainage pipe 200 through the second opening 149 of the lower cover 144 and into the suction bucket 147 of the second monitoring unit 140. Then, open the valve 210 of the drainage pipe 200 to allow seawater inside the suction bucket 147 to be discharged during the subsequent process. Control the crane 410 to lower the monitoring device 100 onto the surface of the seabed soil 900.

Next, please refer to FIG. 4B. When the monitoring device 100 sinks and makes contact with the surface of the seabed soil 900, at this point, control the crane 410 to release the tension of the rope 412. The monitoring device 100 will continue to penetrate into the seabed soil 900 due to its own weight.

Then, referring to FIG. 4C, control the pumping device to start pumping out the water inside the suction bucket 147 to generate suction pressure, allowing the suction bucket 147 to penetrate almost completely into the seabed soil 900. At this point, the rope 412 can be removed.

For the subsequent treatment of drainage pipe 200, two methods can be used. The first method is to close valve 210, maintaining suction pressure in the drainage pipe 200 and the suction bucket. Then, directly connect the drainage pipe 200 to the subsurface float 630 (refer to FIG. 4E) and place it naturally in the sea. The risk of this method is that if the drainage pipe 200 is exposed to unpredictable sea conditions(such as currents and waves), or damage in the future, it may cause the suction pressure inside the suction bucket 147 to disappear, resulting in loosening of the monitoring device 100. However, the relative advantage of this method is that if you want to remove the monitoring device 100 in the future, you only need to perform the reverse operation, which is to pressurize the suction bucket 147 by injecting water through the drainage pipe 200. This will automatically cause the monitoring device 100 to float up from the seabed, making it convenient for recovery.

The second method is to inject a highly fluid and rapidly setting material into the drainage pipe 200, filling the gap between the lower top cover 144 of the suction bucket 147 and the seabed, as well as some sections of the drainage pipe. This blocks the drainage pipe 200 and causes it to fail naturally, maintaining the suction pressure inside the suction bucket 147. This method can avoid the risk of suction pressure failure in the future due to pipeline damage. However, it is unable to decommission the suction bucket 147 by reverse water injection into the drainage pipe 200, as described in the first method. In other words, the difficulty of recovering the monitoring device 100 is higher.

Referencing FIG. 4D, multiple erosion protection works 500 are placed around the monitoring device 100 to reduce the possibility of local scouring caused by sea current disturbances.

Continuing to reference FIG. 4E, the electrical conduit 300 and drainage pipe 200 are fixed to the subsurface float 630, and then connected to the buoy system 600. The buoy system 600 is equipped with a solar photovoltaic system 620 to provide the necessary power for the monitoring device 100. The signal transmission device 640 can transmit data such as seismic wave information, acceleration information, soil pressure information, pore water pressure information, obtained by the monitoring device 100 to the nearby wind turbine 700. Then, the wind turbine 700 uses fiber optics to transmit the aforementioned data back to terrestrial areas for remote monitoring.

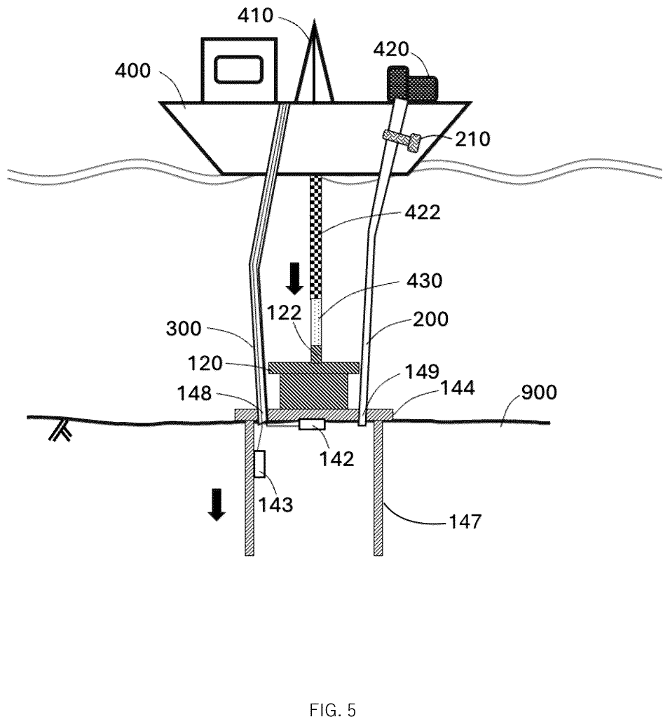

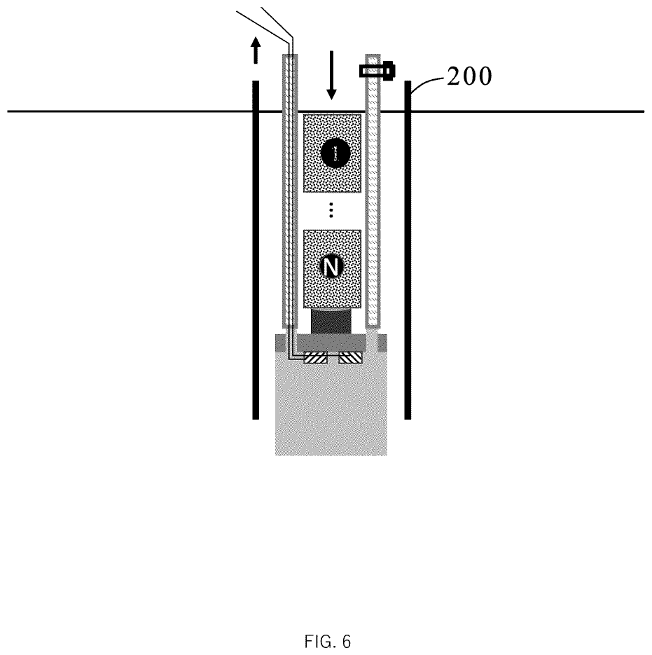

Next, please refer to FIG. 5 for another method of installing the soil monitoring device 100 in the sea area. This method uses the drill rod 422 on the work boat 400, which is connected to the simple joint 122 of the monitoring device 100 through a custom-made drill rod connecting piece 430. Then, one end of the electrical conduit 300 is fixed on the work boat 400, and the other end of the electrical conduit 300 passes through the first opening 148 of the lower top cover 144 to extend into the suction bucket 147 of the monitoring device 100. Next, one end of the drainage pipe 200 is connected to the pumping device 420 on the work boat 400, and the other end of the drainage pipe 200 passes through the second opening 149 of the lower top cover 144 to extend into the suction bucket 147 of the monitoring device 100. Then, the valve 210 of the drainage pipe 200 is opened to gradually drain the seawater from the suction bucket 147. At the same time, the drill rod 422 is controlled to lower the seabed soil liquefaction monitoring device 100 down to the surface of the seabed soil 900. Then, the drill rod 422 is controlled to provide the necessary resistance for penetrating into the seabed soil 900, so that the suction bucket 147 is almost fully penetrated into the seabed soil 900. The subsequent steps of removing the drainage pipe, placing multiple erosion protection works, and connecting the electrical conduit and drainage pipe to the buoy system are similar to those shown in FIGS. 4D and 4E, and will not be further elaborated.

Although the technical content of the present invention has been disclosed in the above paragraph, it is not intended to limit the present invention. Any modifications and improvements made by those skilled in the art without departing from the spirit of the present invention should be encompassed within the scope of the present invention. Therefore, the protection scope of this creation should be determined based on the defined scope of the accompanying patent claims.

1. A seabed soil liquefaction monitoring device for installation on a seabed soil to monitor liquefaction status, the device comprising:
a first monitoring unit exposed above the seabed soil, comprising:
a data logger for recording measurement data of a soil pressure transducer and a piezometer;
an accelerometer for measuring acceleration of the seabed soil liquefaction monitoring device under the action of an earthquake; and
a second monitoring unit fixed to the first monitoring unit and extending into the seabed soil, the second monitoring unit comprising:
a lower cover with a first opening and at least one second opening;
a suction bucket to penetrate the seabed soil, comprising:
the soil pressure transducer for measuring soil stress of the seabed soil; and
the piezometer for measuring pore water pressure of the seabed soil;
wherein the first opening is used for passing through an electrical conduit to accommodate signal lines and power lines of the seabed soil liquefaction monitoring device, while the at least one second opening is used for passing through a drainage pipe to drain water from the suction bucket.

2. The seabed soil liquefaction monitoring device according to claim 1, wherein the suction bucket further includes multiple probes that extend into the seabed soil for connecting to the accelerometer and transmitting seismic wave information data.

3. The seabed soil liquefaction monitoring device according to claim 1, the first monitoring unit is a sealed chamber used to prevent external seawater from entering.

4. The seabed soil liquefaction monitoring device according to claim 1, the at least second opening are set to three and evenly distributed around the lower top cover with the first opening.

5. The seabed soil liquefaction monitoring device according to claim 1, wherein, the piezometer and soil pressure transducer are installed inside the suction bucket and positioned near the first opening.

6. The seabed soil liquefaction monitoring device according to claim 1, wherein, the first monitoring unit further comprising a battery, which provides electric power to the data logger, accelerometer, soil pressure transducer, and piezometer. The battery can store electricity for at least five days' use.

7. The seabed soil liquefaction monitoring device according to claim 1, further comprising a buoy system connecting the electrical conduit and drainage pipe, the buoy system comprising: a signal transmission system for transmitting the data measured from the seabed soil liquefaction device; and a solar photovoltaic system, installed on the buoy system, for generating and transmitting electricity to the battery through the electrical conduit.

8. The seabed soil liquefaction monitoring device according to claim 1, wherein, the first monitoring unit is a first cylindrical shape, and the bottom surface of the first monitoring unit is smaller than the top surface of the second monitoring unit in order to accommodate the first opening and at least one second opening.

9. The seabed soil liquefaction monitoring device according to claim 1, wherein, the first opening and at least one second opening are positioned opposite each other on both sides of the first monitoring unit.

10. The seabed soil liquefaction monitoring device according to claim 8, wherein, the second monitoring unit is a second cylindrical shape, and the circular base area of the second cylindrical shape is larger than the circular base area of the first cylindrical shape of the first monitoring unit.

11. The seabed soil liquefaction monitoring device according to claim 10, wherein, the height of one side of the cylindrical shape of the second monitoring unit is greater than the height of one side of the cylindrical shape of the first monitoring unit.

12. A method for installing a seabed soil liquefaction monitoring device, which is used to penetrate seabed soil liquefaction monitoring device into the seabed soil, the seabed soil liquefaction monitoring device comprising a first monitoring unit and a second monitoring unit, the second monitoring unit comprising a suction bucket formed by a lower top cover and a lower side wall, the installation method comprising:
securing the first monitoring unit to a crane on a workboat by using a rope;
securing one end of an electrical conduit to the workboat, while the other end of the electrical conduit passing through the first opening of the lower top cover and extending into the suction bucket;
connecting one end of a drainage pipe to a pumping device on the workboat, while the other end of the drainage pipe passing through at least one second opening in the lower top cover and extending into the suction bucket;
opening the valve of the drainage pipe;
controlling the crane to lower the seabed soil liquefaction monitoring device down to the surface of the seabed soil;
releasing the tension of the rope to partially penetrate seabed soil liquefaction monitoring device into the seabed soil; and
controlling the pumping device to drain the water in the suction bucket through the drainage pipe, allowing the suction bucket to penetrate almost completely into the seabed soil.

13. A method for installing a seabed soil liquefaction monitoring device, which is used to penetrate the device into the seabed soil, the seabed soil liquefaction monitoring device comprising a first monitoring unit and a second monitoring unit; the second monitoring unit comprising a lower top cover and a suction bucket, the installation method comprising:
connecting a drill rod of a workboat to the first monitoring unit using a rod;
securing one end of an electrical conduit to the workboat, while the other end of the electrical conduit passing through a first opening on the lower top cover and extending into the suction bucket;
connecting one end of a drainage pipe to a pumping device on the workboat, while the other end of the drainage pipe passing through at least one second opening on the lower top cover and extending into the suction bucket;
opening the valve of the drainage pipe; controlling the drill rod to lower the seabed soil liquefaction monitoring device down to the surface of the seabed soil; and
controlling the drill rod to provide a necessary resistance to penetrate the suction bucket into the seabed soil, allowing the suction bucket to penetrate almost completely into the seabed soil.

14. The method for installing the seabed soil liquefaction monitoring device according to claim 12, further comprising:
closing the valve of the drainage pipe to maintain the suction pressure inside the suction bucket when the seabed soil liquefaction monitoring device is almost completely penetrated into the seabed soil; and
connecting the drainage pipe and the electrical conduit to a buoy system.

15. The method for installing the seabed soil liquefaction monitoring device according to claim 13, further comprising:
closing the valve of the drainage pipe to maintain the suction pressure inside the suction bucket when the seabed soil liquefaction monitoring device is almost completely penetrated into the seabed soil; and
connecting the drainage pipe and the electrical conduit to a buoy system.

16. The method for installing the seabed soil liquefaction monitoring device according to claim 12, further comprising:
injecting a highly fluid and fast-setting material into the drainage pipe when the seabed soil liquefaction monitoring device is almost completely penetrated into the seabed soil; and
connecting the drainage pipe and the electrical conduit to a buoy system.

17. The method for installing the seabed soil liquefaction monitoring device according to claim 13, further comprising:
injecting a highly fluid and fast-setting material into the drainage pipe when the seabed soil liquefaction monitoring device is almost completely penetrated into the seabed soil; and
connecting the drainage pipe and the electrical conduit to a buoy system.

18. The method for installing the seabed soil liquefaction monitoring device according to claim 14, the buoy system comprising:
a signal transmission system, which is used for transmitting the data measured by the seabed soil liquefaction monitoring device timely for remote monitoring; and
a solar power system, which is installed on the buoy system for generating and transmitting electricity to the battery through the electrical conduit.

19. The method for installing the seabed soil liquefaction monitoring device according to claim 15, the buoy system comprising:
a signal transmission system, which is used for transmitting the data measured by the seabed soil liquefaction monitoring device timely for remote monitoring; and
a solar power system, which is installed on the buoy system for generating and transmitting electricity to the battery through the electrical conduit.

20. The method for installing the seabed soil liquefaction monitoring device according to claim 16, the buoy system comprising:
a signal transmission system, which is used for transmitting the data measured by the seabed soil liquefaction monitoring device timely for remote monitoring; and
a solar power system, which is installed on the buoy system for generating and transmitting electricity to the battery through the electrical conduit.

21. The method for installing the seabed soil liquefaction monitoring device according to claim 17, the buoy system comprising:
a signal transmission system, which is used for transmitting the data measured by the seabed soil liquefaction monitoring device timely for remote monitoring; and
a solar power system, which is installed on the buoy system for generating and transmitting electricity to the battery through the electrical conduit.

22. The method for installing the seabed soil liquefaction monitoring device according to claim 14, further comprising:
placing multiple erosion protection works around the seabed soil liquefaction monitoring device to reduce the possibility of local erosion caused by sea currents.

23. The method for installing the seabed soil liquefaction monitoring device according to claim 15, further comprising:
placing multiple erosion protection works around the seabed soil liquefaction monitoring device to reduce the possibility of local erosion caused by sea currents.

24. The method for installing the seabed soil liquefaction monitoring device according to claim 16, further comprising:
placing multiple erosion protection works around the seabed soil liquefaction monitoring device to reduce the possibility of local erosion caused by sea currents.

25. The method for installing the seabed soil liquefaction monitoring device according to claim 17, further comprising:
placing multiple erosion protection works around the seabed soil liquefaction monitoring device to reduce the possibility of local erosion caused by sea currents.
